(19) United States
(12) Patent Application Publication
(10) Pub. No.: US 2004/0121416 A1
(43) Pub. Date: Jun. 24, 2004

(54) Assay

(76) Inventor: Frederick John Rowell, Durham (GB)

Correspondence Address: MYERS BIGEL SIBLEY & SAJOVEC, PO BOX 37428, RALEIGH, NC, 27627, US

(21) Appl. No.: 10/469,948

(22) PCT Filed: Mar. 5, 2002
(86) PCT No.: PCT/GB02/00901

(30) Foreign Application Priority Data
Mar. 5, 2001 (GB) 105362.8

(51) Int. Cl. G01N 33/53; G01N 33/537; G01N 33/543; C12M 1/34

(52) U.S. Cl. 435/7.92; 435/287.2

(57) ABSTRACT

There is described an assay system wich comprises; (i) a first dip strip comprising a first material at least partially coated with a repoter converter in an inert carrier; and (ii) a second dip strip comprising a second strip material at least partially coated with a reporter precursor. There is also described an assay system comprising a single foldable or frangible strip and a method of quantitative determination of an assay.

This invention relates to a novel analytical method and to novel apparatus for conducting such methods.

In particular the invention relates to a novel assay and assay apparatus.

Enzyme linked immunoassay (ELISA) was first described in 1971 and since then it has become an important technique in a number of areas, including, diagnostic virology, environmental analysis and forensic analysis. ELISA has replaced a number of more cumbersome serological techniques. Generally, the ELISA techniques comprises the solubilising of antigens, either directly or via an antibody, in an appropriate buffer, the solution then being coated on a plastic surface, e.g. a polystyrene surface. Serum is then added and any antibodies present can attach to the antigen, thereby being bound to the solid phase. The presence or absence of the antibodies can be demonstrated with, for example, anti-human immunoglobulin conjugate or, alternatively, with a conjugate specific to the appropriate antigen. The amount of bound conjugate may be determined by adding an appropriate substrate, such as horseradish peroxidase (HRPO) or alkaline phosphatase. Visual or spectrophotometric methods may then be used to determine a quantitative analysis of the antigen present.

Since the original development of ELISA technology, ELISA has now been employed in the quantitative analysis of other primary binding agents, such as lectins and nucleic acids (RNA and DNA).

The spectrophotometric methods can be conducted employing, for example, wells of microtitre plates or dip strip devices. Generally, the generation of coloured, or fluorescent spots/dots can be achieved using a “reporter material”, for example a molecule that is tagged with molecules that possess the required spectral or light reflecting properties to enable direct observation and/or detection of the intensity of the spot. Examples of such reporter materials are fluorescent liposomes, gold-labelled macromolecules, red blood cells, and latex agglutination attached, for example, to primary or secondary antibodies or avidin.

Alternatively, the “reporter material” can be generated by the employment of an enzyme attached to the primary binding molecules, thus, for example, a substrate (a “converter substrate”) may be added to a reporter precursor, e.g. a reporter enzyme, converting the reporter precursor into a reporter material which possesses the required spectral properties. One of the most commonly used examples of this system employs soluble substrate that produces insoluble coloured products that, once formed, adhere to the material present on the surface within the spot and hence produce a coloured spot.

Assay systems have been described that are based on the use of such enzyme-amplified end points coupled to the direct determination of enzyme activity, such as protease activity, or its use in dot-ELISA systems. The former has been described for an on-filter determination of subtilisin-type enzymes [1] and the latter for beta-lactam antibiotics [2] and estrogenic steroids [3]. In both formats, the end point involves use of alkaline phosphatase and bromochloroindolylphosphate/nitroblue tetrazolium salt (BCIP/NBT), an enzyme-substrate combination that generates an insoluble coloured product that sticks to the surface of a dot or dip strip in the vicinity of the immobilised enzyme. This provides a visual end point for the assay based on the intensity of the resulting spot.

Conventionally with dip strip systems this end point is obtained by dipping an antigen or antibody-coated strip into a solution of the converter substrate or applying a drop of converter substrate to a dot of immobilised enzyme. Such systems are therefore disadvantageous in that they require the preparation and use of a solution of the reporter substrate each time the system is used.

Immunoassays utilise the specific binding capabilities of antibodies or antigens to detect the presence of target molecules in a sample, such as medical diagnostic applications which include a wide variety of analytes in biological fluids such as blood, saliva, and urine.

Several types of immunoassays, useful for distinct applications, already exist. Each such assay type requires a way of distinguishing whether binding sites on an antibody are occupied or free. Typically, this is accomplished by means of a label such as an atom, molecule, enzyme or particle attached permanently to either the antibody or to the analyte or an analog of the analyte.

There is therefore a need for a dip strip assay that is “reagentless”, in that all reagents needed for the assay are pre-prepared and dispensed automatically during the course of the assay.

We have now surprisingly found a novel type of dip strip format that meets this objective.

Thus according to the invention we provide an assay system which comprises

(i) a first dip strip comprising a first strip material at least partially coated with a reporter converter in an inert carrier; and

(ii) a second dip strip comprising a second strip material at least partially coated with a reporter precursor.

In the dip strip of the invention each of the reporter converter material and the reporter precursor is preformulated as a dot or area of reagent at one end of a dip strip.

In use, reporter converter or the reporter precursor may be mixed with the analyte, e.g. a protein or other substance to which the reporter material, e.g. an enzyme-catalysed product derived from the reporter converter and the reporter precursor, sticks.

Any conventionally known reporter converters may be used and the selection of such a converter will vary depending upon, inter alia, the nature of the reporter precursor. Thus, for example, when the reporter precursor is alkaline phosphatase then the reporter converter may be bromochloroindolylphosphate/nitroblue tetrazolium salt (BCIP/NBT). The amount of reporter present may vary, depending, inter alia, upon the nature of the converter, etc. However, the amount of converter may be from 1 to 20% w/v, preferably from 10 to 20% w/v, more preferably 15 to 20% w/v and especially 17% w/v.

In the first dip strip of the invention, the presence of the inert carrier is necessary to retain the reporter converter within the spot, to stabilise the reporter converter within the spot and to amplify the intensity of the coloured spot during the development step. In this format the reporter converter may remain stable under ambient conditions for several weeks. Generally, the inert carrier should be a viscous immobilising agent. The inert carrier may be covalently bonded to the strip material, however, preferentially, the inert carrier is adsorbed onto the surface of the strip material. The inert carrier may be selected form a variety of materials or may comprise a mixture of materials. Such materials include, but are not limited to, sugars and polymeric materials, such as proteinaceous materials. An example of such materials includes gelatin. The amount of inert carrier present may vary, depending, inter alia, upon the nature of the carrier. However, preferred carriers are sugars, such as sucrose. However, the amount of carrier present may vary, depending upon, inter alia, the nature of the carrier, and may be from 0.1 to 10% w/w, preferably 0.5 to 10% w/w, more preferably from 3 to 5% w/w and especially 5% w/w.

In the second dip strip of the invention a dot or area of the dip strip material may be coated with a reagent such as a substrate for the target enzyme covalently linked to a reporter precursor, e.g. a reporter enzyme, an antigen or an antibody. This reporter precursor should be specific for the chemical transformation of the reporter converter immobilised on the first dip strip.

In use, following either direct treatment with the target enzyme as analyte, or with an enzyme-labelled antibody or antigen, the second dip strip is brought into contact with the first dip strip. Thus, the substrate for the reporter precursor, e.g. the reporter enzyme, is wetted due to this contact and now leaches from its spot and makes contact with the reporter converter located on the other surface. The substrate is now converted into insoluble product and sticks to either surface. After a suitable time period the surfaces are separated and the intensities of the resulting spots are determined either visually or via instrumentation such as a scanning densitometer.

In a preferred embodiment of the invention the first and second dip strips may comprise a single strip which is foldable or frangible so as to enable the indicator material to be brought into contact or proximity with the target enzyme/reporter material.

In an especially preferred embodiment the single strip is provided with a foldable region so as to enable the indicator material to be brought into contact or proximity with the target enzyme/reporter material.

In this preferred embodiment, the strip material may, preferentially, have a tacky surface. The tacky surface allows the dip strip to be folded and held together by adhesion of the adjoining tacky surfaces, thus bringing the ends close together, but not in direct contact. The tacky surface may be inherent in the nature of the strip material. Alternatively, the strip material may be coated or at least partially coated with a tacky layer enabling gentle adhesion to occur.

One particular advantage of this aspect of the invention is that, because of the configuration of the strips, no equipment is necessary to force the surfaces of the strip together to effect the development of the spot/coloration.

Generally, the development of the reporter material, by the combining of the reporter converter and the reporter precursor, to produce, for example, a coloured indicator, includes an oxidation step. This oxidation step is disadvantageous because, inter alia, it is time consuming, i.e. it takes 4-5 minutes to develop, furthermore, with the reagentless strips of the invention, the two strips must be disengaged to allow oxidation. Thus, according to a further feature of the invention we have found it advantageous to include an oxidising agent in either reporter converter or the reporter precursor.

The folded ends may be placed into a well of a microtitre plate or its equivalent, containing a small volume of buffer solution. This solution fills the space between the two surfaces by capillary action. After 1-15 minutes (typically 5 minutes) the dip strip is removed and the intensity of the resulting spots noted.

Therefore we further provide an assay system as hereinbefore described wherein an oxidising agent is incorporated into one or both of the reporter precursor and the reporter converter.

In a preferred embodiment the oxidising agent is incorporated in the reporter converter and is therefore immobilised in an inert carrier.

Any conventionally known oxidising agent, or any mixture of oxidising agents, may be used. However, a preferred oxidising agent is hydrogen peroxide. The amount of oxidising agent present may vary, depending, inter alia, upon the nature of the oxidising agent, the reporter material, the inert carrier, etc. However, it is preferred that the oxidising agent is present in an amount of from 0.1 to 4% w/w, preferably 0.5 to 2% w/w and especially 1% w/w.

In a yet further preferred embodiment of the invention, all of the required reagents for an assay may be incorporated into one or a pair of clip strips. In the most preferred embodiment all of the reagents are incorporated into a single dip strip.

Thus, according to a further aspect of the invention we provide a single ELISA strip which comprises a foldable strip material being provided at a first end with a reporter converter and a second end provided with a reporter precursor, wherein the precursor is coated or partially coated with an enzyme. Thus, for example, one end has a spot of a capture antibody on one narrow strip and a narrow strip above this which is impregnated with a solution of pre-mixed capping antibody and alkaline phosphatase-labelled protein A in 5% w/w sucrose solution. The distal end of the strip is coated with the developer (enzyme substrate).

The oxidising agent may be present as an intimate mixture with the reporter converter and/or the reporter precursor. Alternatively, the oxidising agent may be microencapsulated, e.g. in a liposome, such that the oxidising agent is only released when pressure is applied, or when the surface is exposed to detergent in the buffer wetting the other surface, or when the surface is exposed to detergent in the buffer wetting the other surface, for example, by bringing two dip strips together.

In use, with this preferred embodiment of the invention, following either direct treatment with the target enzyme as analyte, or with an enzyme-labelled antibody or antigen, the dip strip is folded to place the two ends of the dip strip together brought into contact with the first dip strip. As hereinbefore described, the substrate for the reporter enzyme is wetted due to this contact and leaches from its spot and makes contact with the reporter enzyme located on the other surface and the substrate is converted into insoluble product and sticks to either surface.

The strip material itself may comprise any conventionally used strip materials.

According to a further feature of the invention we provide an assay kit comprising a first and a second dip strip as hereinbefore described.

Alternatively we provide an assay kit comprising a frangible or foldable dip strip as hereinbefore described.

The assay kit of the invention may also include, for example, a clamp system which is adapted to hold together a pair of dip strips of the invention. In a preferred embodiment the clamp system may be adapted to hold an array of pairs of dip strips.

According to a yet further feature of the invention we provide a method of quantitative determination of an assay which comprises the use of a dip strip as hereinbefore described.

In the method of the invention the pH of the reporter precursor may be varied. However, it is preferred that the pH of the reporter precursor is greater than pH 8.5; preferably between pH 8.5 and 10.5; and especially pH 9.5.

According to the method of the invention the qualitative determination may include a visual colour determination or, preferably, a spectrophotometric determination.

The invention will now be described by way of example only and with reference to the accompanying drawings, in which;

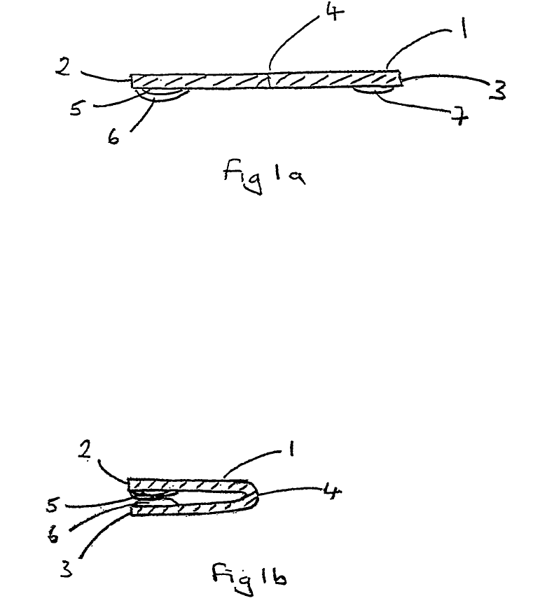

FIGS. 1a) and 1b) are cross-sectional views of a foldable dip strip of the invention;

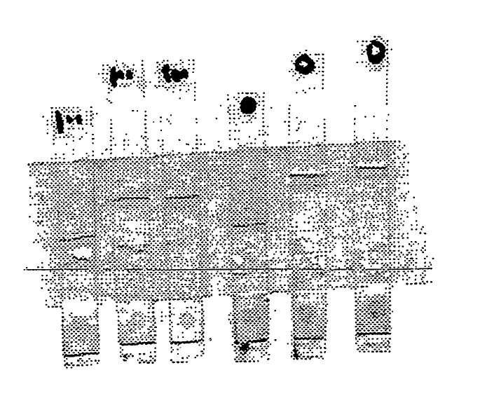

FIG. 2 is a representation of dip strips following development with immobilised BCIP/NBT following the method of Example 1; left to right; first three strips, plus 100 ng subtilisin, next three strips no subtilisin; and

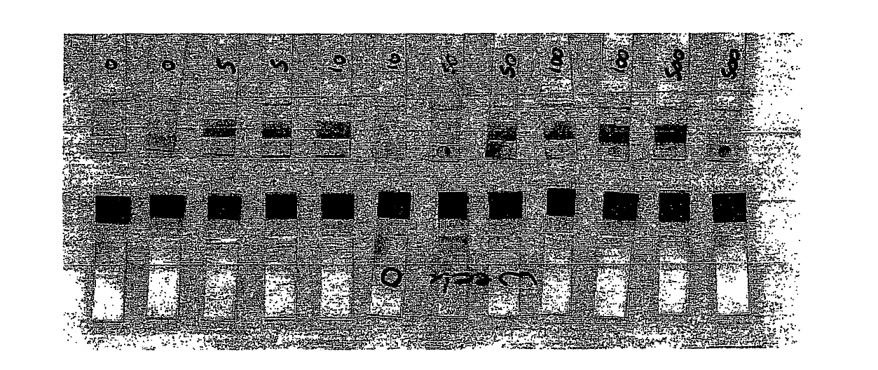

FIG. 3 is a representation of dip strips prepared by the method of Example 4.

Referring to FIGS. 1a and 1b, a dip strip (1) comprises a, e.g. cellulosic material. The strip (1) comprises a first end (2) and a second, distal end (3) and a foldable region (4). On the first end (2) the dip strip (1) is provided with a reporter converter (5) in an inert carrier (6) and at the second end (3) the dip strip (1) is provided with a reporter precursor (7).

In use, the reporter precursor (7) is brought into contact with an analyte and then the dip strip is folded about the foldable region (4) so as to bring the reporter converter (5) and the reporter precursor (7) into contact with one another.

### EXAMPLE 1

Immobilised Reporter Precursor

A reporter precursor composition was prepared according to the following formula.

| | | |
|---|---|---|
| Hydrogen peroxide | 50 | μl |
| BCIP/NBT | 200 | μl |
| Gelatin | 50 | μl |

The components were mixed together and dissolved in water (5 ml).

### EXAMPLE 2

Determination of Subtilising Using a Dip Strip Assay

3-Bromo-4-chloroindolylphosphate (BCIP) substrate was dissolved in 1% (w/v) of gelatin and the tip of a cellulose nitrate covered dip strip was immersed in this solution. The strip was immediately removed and the surface allowed to air dry over 15 min. It was stored in a covered plastic box until used. A second dip strip was prepared with a small volume of alkaline phosphatase-labelled anti-donkey anti-rabbit antibody reagent. This was immersed in a solution of citrate buffer (500 μl of 0.1 M, pH 9.5) either with 100 ng of subtilisin enzyme (+) or without any enzyme (−). After 15 minutes at ambient temperature, the dip strip was removed and the first dip strip with the substrate dot brought into contact with the second wet strip such that the two spots of antibody and substrate were in contact. After 5 minutes the strips were separated and the intensities of the spots on each surface examined visually and the intensities determined using an optical scanner.

The intensity of the spots on the surface of the strips was determined using an optical scanner for the strips where the substrate was initially immobilised. This was less for those strips that had been in contact with subtilisin [intensity 127±36.1 (n=3)] than for the corresponding spots on the three strips that had been in contact with 100 ng of subtilisin [246±10.6 (n=3)]. The spots are shown below following optical scanning of the developed strips.

It was observed that spot development was not complete during the contact of the surfaces and was accelerated when the surfaces were subsequently exposed to air. In order to simulate such oxidation, hydrogen peroxide and urea hydrogen peroxide were added to the solution into which the surface with the immobilised reporter attached is immersed, or they were incorporated into the solution of the reporter precursor during the coating process. Using this approach full development of the coloured spot was observed on separating the two surfaces in contact. It has also been found that the end point with alkaline phosphatase and BCIP/NBC in this format is pH dependent with colour only developing at pH above 8.5.

### EXAMPLE 3

Dip Strip Immunoassay for 17-β-estradiol (ED)

One set of dip strips was prepared by spotting 1 μL of a solution of 17-β-estradiol-bovine serum albumin (ED-BSA) at a concentration of 1 μg/ml in coating buffer at the centre of a small square of cellulose nitrate attached to a cellulose acetate strip [3]. The tips of a second set of similar strips were immersed in a solution consisting of 1% by volume hydrogen peroxide (50 μL), 4% (w/v) of BCIP/NBT and 1% aqueous gelatin (final concentrations) for 10 seconds then removed. They were allowed to dry for 10 minutes at ambient temperature and then again dipped into the coating solution. The process was repeated so that three coatings were obtained. The ends of the first set of tips were immersed in a solution of rabbit anti-ED (1000 μl) located within 10M-type personal monitors [3]. After shaking the filters for 15 minutes the strips were removed, washed under the tap and immersed in a solution of alkaline phosphatase-labelled anti-rabbit second antibody for 15 min. The strips were then again washed under the tap and immersed in either phosphate buffer or glycine buffers at pH 7.5, 8.5 or 9.5. The coated ends of the second set of strips were then brought into contact with the wet surface of the first strips. This was achieved by placing them on the surface of a glass plate and covering them with a second plate. After 10 minutes the strips were separated and the colour intensities of the spots on the surface of the ED-BSA-coated strips noted. The intensity values were significantly darker for the pH 9.5 buffer (p<0.001), with a value of 219, than the pH 8.5 (value of 254) and pH 7.5 (value of 255) although a slight colour was visually observed with the pH 8.5 buffer.

The experiment was repeated using only the pH 9.5 glycine buffer but comparing strips that contained hydrogen peroxide within the gelatin coating with those that had none of this oxidising agent. It was noted that blue spots were present on separating the two strips when hydrogen peroxide was present whilst in its absence no corresponding spots were initially observed but developed some 5-10 minutes later. Further experiments have demonstrated that the activity of the hydrogen peroxide persists for several days following the coating of the strips with the gelatin-substrate.

### EXAMPLE 4

Self Contained Dip Strip Assay for Savinase

Materials Required

Acetate plate sealers (Cat No. 3501, Dynex)

φ-savinase antibodies raised in rabbit (in-house, R1-B4)

Bovine Serum Albumin (BSA) (Sigma A-7906)

1.0 μm cellulose nitrate filter paper, 47 mm diameter (Whatman, Cat No. 7190-004)

Coating buffer

Deionised water

Dip strip substrate buffer (see Example 5)

Fast, hardened filter paper for retention of course and gelatinous precipitates/‘Filter Paper 54’ (Whatman, Cat No. 90904)

Microtitre plate (immulon 4HBX, Dynex)

Phosphate buffered saline with azide

Protein A (Sigma) P-6031)

Protein A—Alkaline phosphatase conjugate (Sigma P-9650)

Tween 20 (Sigma P-7949)

Sucrose (Sigma S-9378)

Savinase (Novo Nordisk, Batch 15-1195)

Buffers

Preparation of Phosphate Buffered Saline (with Azide) Containing 0.1% (^{v}/ᵥ) Tween 20 and 0.1% (^{w}/ᵥ) BSA (1 l), pH 7.2-7.4 (PBST+0.1% BSA)

1. Add 1 ml of Tween 20 to 1 l of phosphate buffered saline with azide.

2. Add 1 g of BSA to the 1 l to this solution.

3. Check the pH to ensure it is between 7.2-7.4.

Preparation of 5% (^{w}/ᵥ) Sucrose Buffer

1. To make 10 ml of 5% sucrose buffer, dissolve 500 mg of sucrose into 10 ml of deionised water.

Method

Prepare solutions as follows:

Preparation of Pre-mix (for 32 Dip Strips for Standard Curve), (1:100, 1:250 K)

1. Dilute 5 μl of R1-B4 (rabbit antiserum) and 2 μl of protein A-alkaline phosphatase conjugate in 500 μl of PBST+0.1% BSA and mix well.

2. Leave to equilibrate at room temperature for at least 2 hours.

Preparation of Substrate Dip Strips

1. Cut acetate plate sealers into strips so that length is approximately 3.5 cm.

2. Remove 1.3 cm of backing from bottom of acetate plate sealers (what is left is used as a label).

3. Cut Fast, hardened filter papers for retention of course and gelatinous precipitates/‘Filter Paper 54’ into strips 0.5 cm in width.

4. Place strips of filter paper at bottom of acetate strips.

5. Cut the long acetate strips into individual strips of 0.5 cm width.

6. Prepare substrate for self contained dip strip assay by adding 140 μl of dip strip substrate to 280 μl of 5% sucrose buffer and vortex for 10 seconds.

7. Pipette 7 μl of the substrate mixture onto the surface of each dip strip and allow to dry, for at least 2 hours at room temperature.

Preparation of Antibody Coated Dip Strips

1. Cut acetate plate sealers into strips so that length is approximately 3.5 cm.

2. Remove 1.3 cm of backing from bottom of acetate plate sealers (what is left is used as a label).

3. Cut cellulose nitrate filter paper into strips 0.3 cm in width.

4. Place strips of cellulose nitrate filter along the bottom of the acetate strips.

5. Cut another set of fast hardened filter papers for retention of course and gelatinous precipitates/‘Filter Paper 54’ into strips 0.3 cm in width.

6. Place strips of fast, hardened filter paper 0.1 cm above the top of the cellulose nitrate filter already laid onto the acetate dip strip.

7. Cut long acetate strips into Individual dip strips of 0.5 cm width.

8. Prepare a solution of protein A at a concentration of 10 μg ml⁻¹ in coating buffer.

9. Place a 1 μl spot of 10 μg ml⁻¹ protein A onto the filter of each dip strip.

10. Incubate at room temperature for 10 minutes.

11. Rinse the filters of the dip strips in deionised water for 5 seconds, and leave to dry for 15 minutes at room temperature.

12. Prepare a solution of (1:2 K) φ-savinase antibody solution, incubate at Room Temperature for 30 minutes.

13. Once the filters have dried, immerse the dip strips in 250 μl of 1:2 K φ-savinase antibody solution, incubate at Room temperature for 30 minutes.

14. Rinse the filters of the dip strips in deionised water for 5 seconds, and leave to dry at room temperature for 15 minutes.

15. Once the filters have dried thoroughly pipette 4 μl of the premix solution onto the fast, hardened filter paper section of the dip strip, and allow to dry at room temperature for 30 minutes.

Self Contained Assay for Savinase

1. Weigh 42.6 mg of savinase and dissolve in 1 ml of deionised water to give a stock solution of 1 mg ml⁻¹.

2. Prepare standards from this stock in PBST+0.1 BSA (Typically 200 ng ml⁻¹-2 ng ml⁻¹).

3. Place antibody coated dip strips into 250 μl of the appropriate savinase standard and incubate at room temperature for 30 minutes, and rinse in deionised water.

4. While the dip strips are still wet, dip the substrate dip strips into substrate buffer to wet the filter.

5. Stick the two dip strips together so that the filters are facing each other, and place them into a well of a microtitre plate containing 25 μl of substrate buffer. This ensures a liquid phase between the two dip strips allowing development.

6. Incubate for 10-15 minutes until spots develop, then separate the dip strips and wash with deionised water.

7. Leave to dry in the dark at room temperature.

8. Once the filters are dry scan the dip strips into PaintShop Pro and save as *.bmp file.

9. Note the intensities of the spots using the PC software package BANDLEADER (square is drawn inside spot).

A representation of dip strips prepared by the method of this example is provided in FIG. 3 (numerals are ng/ml).

### EXAMPLE 5

Preparation of Dip Strip Substrate

Materials Required

5-Bromo-4-chloro-3-indoyl phosphate (Sigma B-8503)

Dimethyl formamide (DMF)

Deionised water

Nitroblue Tetrazolium (Sigma N-6876)

Substrate buffer

Buffers

Preparation of Dip Strip Substrate

1. Dissolve 50 mg of 5-Bromo-4-chloro-3-indoyl phosphate and 75 mg of Nitroblue Tetrazolium into 1 ml of dimethyl formamide.

2. Add 2-4 drops of deionised water.

3. Sonicate for 10 min.

4. Store at 4° C. in the dark.

5. When required, dilute with substrate buffer−1 part in 30 (BCIP/NBT stock: substrate buffer). (Note prepare only 10 mins before required).

1. An assay system which comprises;
(i) a first dip strip comprising a first strip material at least partially coated with a reporter converter in an inert carrier; and
(ii) a second dip strip comprising a second strip material at least partially coated with a reporter precursor.

2. An assay system according to claim 1 characterised in that the reporter converter material and the reporter precursor are each preformulated as a dot or area of reagent at one end of a dip strip.

3. An assay system according to claim 1 characterised in that the amount of reporter converter material present is from 1 to 10% w/w.

4. An assay system according to claim 1 characterised in that Thus, for example, when the reporter precursor is alkaline phosphatase then the reporter converter may be bromochloroindolylphosphate/nitroblue tetrazolium salt (BCIP/NBT).

5. An assay system according to claim 1 characterised without the amount of inert carrier present is from 0.1 to 4% w/w.

6. An assay system according to claim 1 characterised in that the inert carrier is a polymeric material.

7. An assay system according to claim 1 characterised in that the inert carrier is a proteinaceous material.

8. An assay system according to claim 1 characterised in that the carrier is gelatin.

9. An assay system according to claim 1 characterised in that the carrier is selected from the group gelatin, a monosaccharide, a disaccharide and a polysaccharide.

10. An assay system according to claim 1 characterised in that one or both of the reporter converter and the reporter precursor also comprises an oxidising agent.

11. An assay system according to claim 10 characterised in that the oxidising agent is incorporated into the reporter converter.

12. An assay system according to claim 11 characterised in that the oxidising agent is present at a level of from 0.1 to 4% w/w.

13. An assay system according to claim 10 characterised in that the oxidising agent is hydrogen peroxide.

14. An assay system according to claim 1 characterised in that the first and second dip strips may comprise a single strip which is foldable or frangible so as to enable the reporter converter material to be brought into contact or proximity with the reporter precursor.

15. An assay system according to claim 14 characterised in that the single strip is provided with a foldable region so as to enable the reporter converter material to be brought into contact or proximity with the reporter precursor material.

16. An assay system according to claim 14 characterised in that the strip material has a tack surface.

17. An assay system according to claim 16 characterised in that the strip material at least partially coated with a tacky layer.

18. An assay system according to claim 14 characterised in that the system has all the necessary reagents incorporated on a single strip.

19. An assay system according to claim 18 characterised in that the system comprises a single strip a first end of which is provided with a reporter converter and a carrier and a second end is provided with a reporter converter and a carrier and a second end is provided with a reporter precursor and a carrier, overlayered with an enzyme.

20. An assay kit comprising a first and a second dip strip according to claim 1.

21. An assay kit comprising a frangible or foldable dip strip according to claim 14.

22. An assay kit according to either of claims 20 or 21 characterised in that the kit also includes a clamp system.

23. A method of quantitative determination of an assay which comprises the use of a dip strip according to claims 1 or 14.

24. A method according to claim 23 characterised in that the pH of the reporter precursor is pH>8.5.

25. A method of qualitative determination of an assay which comprises the use of a dip strip according to claims 1 or 14 followed by spectrophotometric colour determination.

26. An assay system or a method substantially as described with reference to the accompanying examples.
